(19) United States
(12) Patent Application Publication
(10) Pub. No.: US 2011/0286941 A1
(43) Pub. Date: Nov. 24, 2011

(54) COMBINATION OF TWO MATERIAL COMPOSITIONS FOR AN ORAL HYGIENE PRODUCT

(76) Inventor: Klaus-Uwe Busch, St. Johann Im Pohgau (AT)

(21) Appl. No.: 13/129,923

(22) PCT Filed: Nov. 17, 2009
(86) PCT No.: PCT/EP2009/065318

§ 371 Date: Aug. 8, 2011

(30) Foreign Application Priority Data
Nov. 18, 2008 (DE) 10 2008 043 818.9

(51) Int. Cl. A61K 8/21; A61C 15/04; B65D 85/00; A61K 9/68; A61Q 11/00

(52) U.S. Cl. 424/48; 424/52; 206/524.1; 132/321

(57) ABSTRACT

The present application relates to a combination of two separately stored and jointly used compositions, which are suitable for the combat and the prevention of caries. Thereby, each of the two compositions respectively comprises a fluorine compound. Further, one composition comprises an acidic pH-value, while the other composition comprises a basic pH-value.

### FIELD OF THE INVENTION

The present invention relates to a combination of two separately stored and jointly used or mixed compositions, which may be used in an oral hygiene product. This combination of two compositions is especially suited for combating and preventing of caries.

### BACKGROUND

Caries is a dental disease, which is widespread, especially in industrialized countries. The caries process damages the enamel of teeth and leads at an advanced stage to lesions of the teeth, which have to be treated by removing the tooth substance and filling the cavity with filling material.

Decisive for the caries process are the bacteria contained in the biofilm of the teeth, as for example streptococcus mutans. These bacteria convert the carbohydrates contained in the biofilm, especially the sucrose resulting from sugar consumption, into acids, wherein the corresponding bacteria further spread in the acidic environment (self-enhancing process). The H⁺ ions contained in the acids attack the surface of the enamel, which mainly consists of hydroxylapatite (HAP). The H⁺ ions dissolve the HAP of the enamel surface, such that calcium ions (Ca²⁺ ) as well as carbonate and phosphate ions are set free, which diffuse into the biofilm. This process is called demineralization of the enamel. When the pH-value of the biofilm increases again, i. e. when the biofilm is less acidic, the Ca²⁺ and phosphate ions may again combine to HAP, which is deposited at the enamel surface (remineralisation of the enamel). There is a balance between the two processes demineralization and remineralization of the enamel in a healthy tooth such that the enamel is maintained.

If fluorine is contained in the biofilm, the fluorine combines especially quickly with the dissolved phosphate to form fluorapatite (FAP), which may also be incorporated into the enamel: In this manner, the remineralization of the tooth surface is strengthened. Advantageously, the FAP has, in comparison to HAP, a greater acid resistance, which makes the tooth surface more resistant with regard to the caries process (“Hardening of the tooth”). Thus, fluorine acts positively in double regard: Firstly, the F⁻ ions react especially easy with phosphate to FAP, which secondly is also especially acid resistant. Further, the pH-value of the biofilm plays a significant role in the formation of caries since the H⁺ ions of the acids effect the demineralization of the enamel.

The progress made in the past thirty years in the combat against caries is mainly due to the increased application of fluorides, for example in tooth pastes. Nevertheless, when using fluorine containing oral hygiene products, a significant caries risk remains, such that caries still occurs very frequently.

It is thus the object of the present invention to combat caries more effectively than in the past.

### SUMMARY OF THE INVENTION

This object is solved by a combination of two separately stored and jointly used or mixed compositions, characterized in that both compositions respectively comprise a fluorine compound; and the one composition comprises an acidic pH-value, while the other composition comprises a basic pH-value.

As confirmed by a clinical trial conducted by the applicant, which is described in detail below, a tooth paste with two components according to one embodiment of the present invention, in which the one component comprises fluorine in a basic environment and the other component comprises fluorine in an acidic environment, decreases the risk of caries by 80 percent, if one takes as a comparison a conventional tooth paste with a fluoridic composition. This strong effectivity of the tooth paste with two components shows that the acidic component synergistically cooperates with the basic component. This mutual enhancing of the two components among themselves is very surprising and indicates that inhomogenities of the pH-value in the biofilm accelerate the reaction of the fluorine with the calcium and the phosphate ions and facilitate the incorporation of the FAP into the enamel, respectively.

It is important for the inventive combination of two compositions that the two compositions are stored with a different pH-value until their application for the dental cleaning since otherwise their pH-values would at least partly balance out themselves and thus, the desired inhomogenities with regard to the pH-value would not occur. During the application in the mouth, the two components are brought into contact with each other—in particular they are mixed—wherein no specific degree of mixing has to be reached.

Preferably, the concentration of the fluorine compound in the two compositions of the inventive combination lies between 0.02 and 1 percent by weight (respectively 100 to 5000 ppm) and particularly preferred lies between 0.04 percent by weight and 0.28 percent by weight (respectively 200 to 1400 ppm). These concentrations of the fluorine compound take into consideration the medical concerns, which exist with regard to very high fluorine concentrations.

Apart from that, the pH-value of the composition with acidic pH-value preferably lies between 5.3 and 6.5, while the pH-value of the basic composition lies between 7.8 and 8.8. It was found by the applicant that such pH-values enable a good cooperation of the two compositions and at the same time a good medical compatibility of the respective oral hygiene product.

In preferred embodiments, the inventive combination of two compositions is used in an oral hygiene product, particularly in a tooth paste with two components, an oral rinse with two components, a dental floss or a tooth cleaning chewing gum. According to the invention, also containers with two separate chambers are provided, which may receive the two described compositions until their joint use. No specific requirements are necessary for the container or its chambers. It is sufficient that the chambers substantially prevent the mixing of the compositions until their application. The separation of the chambers may therefore be realized by different measures, respectively means. Further, it is conceivable that the two compositions are filled into one chamber, wherein stabilizers or thickening agents ensure that the two components do not mix in the chamber.

### DETAILED DESCRIPTION OF THE INVENTION

The applicant has carried out an 8 year clinical trial, in which the effect of different tooth pastes on the frequency of caries has been examined, wherein the different tooth paste respectively contained different substances or combinations of substances.

In this medical trial, there were all together four groups: Each of the groups consisted of 50 percent men and of 50 percent women. Further, the probands were chosen and assigned such that in the beginning of the trial equivalent test groups resulted with regard to the oral condition of the group members, the general health of the group members, the age as well as the mental capabilities of the group members, to at least carry out an average oral hygiene.

The oral hygiene of the participants of the study was standardized by intensive oral hygiene training. Before the start of the study, each proband received a training for a professional tooth cleaning and, except from the tooth paste, the same oral hygiene material (tooth brush).

All test groups were given the same basic tooth paste. The basic tooth paste of the study consisted of water, humectants, an abrasive, aroma, sweetener (saccharin). Only one test group used a tooth paste (tooth gel) with the double fluorine system according to the invention. The toothpaste of the individual test groups differed in that the basic tooth paste was provided with different fluorine compounds (which are currently known as the most effective ones) and in that the basic tooth paste (i. e. the carrier formulation) was provided with three different pH-values. For the pH-values an acidic, a basic and a neutral pH-value were chosen, respectively. According to the latest scientific standards, there is no clinical superiority of the individual fluorine compounds.

In the following, the tooth paste for the 8 groups is given in detail:

Group 1: Only basic tooth paste without fluorine

Group 2: Addition of sodium fluoride to the basic tooth paste; pH-value: 5.4

Group 3: Addition of sodium fluoride to the basic tooth paste; pH-value: 8.5

Group 4: Addition of sodium fluoride to the basic tooth paste; pH-value: 7.0

Group 5: Addition of amine fluoride to the basic tooth paste; pH-value: 5.4

Group 6: Addition of amine fluoride to the basic tooth paste; pH-value: 8.5

Group 7: Addition of amine fluoride to the basic tooth paste; pH-value: 7.0

Group 8: Two-component tooth paste with a pH-value of the first component of 5.9 and a pH-value of the second component of 8.3, wherein both components contain sodium fluoride. However, comparable results are achieved if the fluorine compound is amine fluoride.

Here, the respective tooth pastes were provided with the following concentrations of the agents: sodium fluoride 0.24 percent by weight (1200 ppm), amine fluoride 0.24 percent by weight (1200 ppm). In group 8, two equally long strips of tooth paste were applied onto the tooth brush. Hereby, optionally, two different tooth paste tubes or one tube with two containers may be used. Preferably, the two tooth pastes are only mixed in the oral cavity itself, i.e. not already at the outlet of the tube.

It goes without saying that also tooth pastes with other concentrations of fluorine compounds may be preferred under certain circumstances and therefore belong to the subject matter of the invention. However, at least 0.02 percent by weight (100 ppm) fluorine should be contained in the individual components.

The risk of caries was surveyed by measuring the number of new carious teeth in relation to control group 1, wherein the control groups merely applied the basic tooth paste. All probands of groups 1-8 were examined once per quarter, i. e. 4 times per year, for an analysis. All of the above described measuring methods for tooth diseases were standardized and did not vary over the entire duration of the trial. The measurements of new carious teeth in group 1 were taken as reference measure (100 percent).

As a result of the trial, it was found for the risk of caries in detail:

A tooth paste with the addition of fluorine in the groups 2 to 7 (sodium fluoride or amine fluoride) with an arbitrary pH-value could reduce the risk of caries with regard to group 1 (without fluorine) by 20 to 30 percent. This result lies within the frame of previous trials for tooth pastes with the addition of sodium fluoride as well as amine fluoride. Surprisingly, the addition of fluorine by means of the described two-component system (group 8) reduced the risk of caries by up to 80 percent with regard to groups 2 to 7. This means that in group 8, the risk of caries was decreased with regard to group 1 (basic tooth paste) even by approximately 86 percent. Thus, a significant synergistic effect of the two fluorine containing components in a two-component system (acidic/basic) was shown, which goes far beyond the effect of fluorine alone in all previously used conventional forms.

### CONCLUSIONS

Altogether, a tooth paste with the combination of fluorine in two components (acidic/basic) could significantly decrease the risk of caries (by 80 percent, cf. above). This large reduction of the risk of disease was surprising since the agent fluorine alone which is given in 6 different dosage forms (groups 2 to 7) is by far not as effective, and such a big synergistic effect could be expected.

Although in the trial described here, the combination of two fluorine components (acidic/basic) was applied in a tooth paste, the application of two fluorine components (acidic/basic) in an oral rinsing, a dental floss, a chewing gum or a tooth cleaning chewing gum is also envisioned since, here, similar synergistic effects between the two components can be expected.

1. Combination of two separately stored and jointly used compositions, characterized in that
a. each of the two compositions respectively comprises a fluorine compound; and
b. one composition comprises an acidic pH-value and the other composition comprises a basic pH-value.

2. Combination according to patent claim 1, characterized in that the concentration of the fluorine compound in both compositions lies between 0.02 percent by weight and 1 percent by weight.

3. Combination according patent claim 2, characterized in that the concentration of the fluorine compound in both compositions lies between 0.04 percent by weight and 0.28 percent by weight.

4. Combination according to one of patent claims 1 to 3, characterized in that the pH-value of the composition with the acidic pH-value lies between 5.3 and 6.5.

5. Combination according to one of the patent claims 1 to 4, characterized in that the pH-value of the composition with the basic pH-value lies between 7.8 and 8.8.

6. Oral hygiene product with two components characterized in that one component respectively includes one of the two separately stored compositions according to one of claims 1 to 5.

7. Tooth paste with two separately stored phases, characterized in that one phase respectively includes one of the two separately stored compositions according to one of claims 1 to 5.

8. Oral rinse with two separately stored components, characterized in that one component respectively includes one of the two separately stored compositions according to one of claims 1 to 5.

9. Dental floss with two components, characterized in that one component respectively includes one of the two separately stored compositions according to one of claims 1 to 5.

10. Chewing gum for dental cleaning with two components, characterized in that one component respectively includes one of the two separately stored compositions according to one of claims 1 to 5.

11. Use of a combination of two separately stored compositions according to one of claims 1 to 5 for the treatment of caries.

12. Use of a combination of two separately stored and jointly used compositions according to claim ii, wherein the two compositions are only mixed in the oral cavity.

13. Container with a first and a second chamber, wherein the first chamber contains a composition with a fluorine compound in an acidic environment and wherein the second chamber contains a composition with a fluorine compound in a basic environment.

14. Container according to patent claim 13 with compositions according to one of patent claims 2 to 5.
